SEQUENCE LISTING (9 sequences)

SEQ ID NO: 1 - DNA, 150 bp, artificial sequence
  TBE cassette
gataagcttcctccgtcccccgcccgccgcgccatgcctgtggccggctcgtcagtcaggctagctatagcggacttgggcccagtttatggcttccagtggaggcattttggggcagaatacagagatatggaatcagattaagcttgc

SEQ ID NO: 2 - DNA, 42 bp, artificial sequence
  TBE1 in the forward orientation
cctccgtcccccgcccgccgcgccatgcctgtggccggctcg

SEQ ID NO: 3 - DNA, 71 bp, artificial sequence
  TBE2 in the forward orientation
ggacttgggcccagtttatggcttccagtggaggcattttggggcagaatacagagatatggaatcagatt

SEQ ID NO: 4 - DNA, 42 bp, artificial sequence
  TBE1 in the reverse orientation
gctcggccggtgtccgtaccgcgccgcccgccccctgcctcc

SEQ ID NO: 5 - DNA, 71 bp, artificial sequence
  TBE2 in the reverse orientation
ttagactaaggtagagagacataagacggggttttacggaggtgaccttcggtatttgacccgggttcagg

SEQ ID NO: 6 - DNA, 21 bp, artificial sequence
  PCR sense primer for TS mRNA
tttggacagcctgggattctc

SEQ ID NO: 7 - DNA, 21 bp, artificial sequence
  PCR antisense primer for TS mRNA
aaagcaccctaaacagccatt

SEQ ID NO: 8 - DNA, 21 bp, artificial sequence
  PCR sense primer for beta-actin
cagctcaccatggatgatgat

SEQ ID NO: 9 - DNA, 18 bp, artificial sequence
  PCR antisense primer for beta-actin
ccagacgcaggatggcat

(19) United States
(12) Patent Application Publication
(10) Pub. No.: US 2004/0161781 A1
(43) Pub. Date: Aug. 19, 2004

(54) Compositions and methods for identifying thymidylate synthase modulators

(76) Inventors: Bruce J. Dolnick, Orchard Park, NY (US); Qi Wu, Buffalo, NY (US); Edward Chu, Orange, CT (US); Xiukun Lin, New Haven, CT (US)

Correspondence Address: HODGSON RUSS LLP, ONE M & T PLAZA, SUITE 2000, BUFFALO, NY, 14203-2391, US

(21) Appl. No.: 10/719,580

(22) Filed: Nov. 21, 2003

Related U.S. Application Data
(60) Provisional application No. 60/428,345, filed on Nov. 22, 2002.

(51) Int. Cl. C12Q 1/68

(52) U.S. Cl. 435/6

(57) ABSTRACT

The present invention provides compositions and methods for screening of compounds that decrease the activity of thymidylate synthase either directly or indirectly. The composition of the invention comprises a DNA construct comprising from the 5′ to the 3′ direction, a TBE cassette comprising one or more copies, in forward or reverse orientation, of TBE1 and TBE2, and a reporter gene operably linked to a promoter and optionally a selectable marker. The method of the invention comprises growing cells comprising the construct of the invention in the presence or absence of a test compound such that a test compound which decreases the activity of

This application claims priority to U.S. Provisional Application No. 60/428,345 filed on Nov. 22, 2002, the disclosure of which is incorporated herein by reference.

This work was supported by NIH grants CA91114 and CA16056. The government has certain rights in the invention.

### FIELD OF THE INVENTION

This invention relates generally to the area of inhibitors of cell proliferation and more particularly to a method and compositions for rapid screening and identification of inhibitors of cell proliferation.

### BACKGROUND OF THE INVENTION

Thymidylate Synthase (TS) is an essential enzyme involved in the synthesis of the deoxyribonucleotide thymidine 5′ monophosphate (dTMP) from the deoxyuridine 5′-monophosphate (dUMP)[Danenberg, 1977]. The deoxyribonucleotide dTMP is a critical precursor for the synthesis of DNA. Since this enzyme is required for DNA biosynthesis and repair, it has become a common target for anticancer agents. Inhibition of TS or its down regulation is a major goal in cancer chemotherapy. This is attested to by the number of TS inhibitors introduced over the last decade by the pharmaceutical industry and the continued use for over forty years of the drug 5-fluorouracil, a prodrug for the inhibition of TS. More recently developed inhibitors include Ralitrexed® and Tegafur®. These compounds have been used for the treatment of solid tumors including gastrointestinal, breast, head and neck, colorectal and ovarian.

The importance of TS as a target in cancer chemotherapy has been further suggested by the demonstration that TS levels can be a partial predictor of the clinical outcome of treating cancers with TS inhibitors in patients with stomach and colon cancer. In studies with these two types of cancers, patients who had low TS levels tended to respond to treatment and had longer survival times compared to patients with higher levels of TS [Leichman, 1997; Lenz, 1995].

The regulation of TS appears to include control of the synthesis and degradation of the enzyme and the RNA species that are involved in coding for the enzyme, as well as regulation of translation of TS mRNA. Evidence has been published that implicate transcriptional regulation of the TS gene as well as post-transcriptional regulation as contributing to the amounts of TS [Li, 1995; Johnson, 1994].

The level of TS within tumor cells is correlated with the ability of cells to proliferate and to overcome cell death induced by treatment with TS inhibitors. Because of this, lowering the amount of TS within tumor cells is a desirable goal for cancer treatment. Although a number of effective TS inhibitors are currently known and used in the treatment of neoplastic disease, the further manipulation of TS levels by targeting regulatory pathways that control the level of TS within the cell is not currently feasible, but is highly desirable as an adjunct to improving the response of tumors to TS inhibitors. The current inability to lower TS levels arises from current limitations in identifying drugs that affect TS levels and because of a current lack of understanding of the cellular mechanisms regulating TS levels.

In a series of reports beginning in 1991, Chu et al. reported the discovery that TS could bind to specific binding elements (TBEs—TS Binding Elements) present in some mRNAs with high affinity and interfere with translation of the mRNAs to protein[Chu, 1991; Chu, 1993; Chu, 1994; Chu, 1995; Chu, 1996; Chu, 1996; Chu, 1999; Lin, 2000]. TS mRNA was found to have two of these TBEs, one in the 5′-noncoding region and 6 initial amino acids of protein-coding region and one in the protein-coding region. In separate reports the TBEs were inserted into recombinant plasmids that expressed the light producing protein luciferase. Binding of TS to a TBE upstream of the mRNA region coding for luciferase protein was shown to inhibit luciferase when the recombinant mRNA was expressed in tumor cells. This inhibition of luciferase production could be monitored by light produced by luciferase. Thus, cells containing a plasmid (or multiple copies of a plasmid) expressing luciferase mRNA with a TBE produced low amounts of light, but addition of TS inhibitors to these cells caused the cells to produce more light. The increased production of light was found to be due to the binding of the inhibitors or their metabolites to TS with the subsequent loss of ability of TS to bind to the TBE in the luciferase mRNA. This removal of TS from luciferase mRNA resulted in increased production of luciferase protein and consequently an increase in light production. Thus the recombinant luciferase mRNAs were used as tools to demonstrate changes of the interactions of TS with TBEs.

Currently, no reliable tools are available for rapid screening and identification of compounds that can modulate TS levels. The construct of Lin et al. [2000] only contains the TBE from the protein coding region of TS (thymidylate synthase mRNA) and not the TBE from the 5′-UTR (untranslated region). Therefore, the construct is unable to be responsive to any agents that affect either TS binding to the 5′-UTR TBE or affect any gene products that affect the ability of TS to bind to that specific element. The Lin et al. construct therefore does not provide a screening tool for identification of agents that affect TS through a wide variety of mechanisms. Further, this construct does not contain a selectable marker. Therefore, it is difficult to transfect a wide variety of cells with this plasmid and obtain cell lines that have a stable phenotype with respect to the reporter. Co-transfection of this reporter plasmid with another carrying a selectable marker may yield transfected cells, but the stability of the reporter in the transfected cells is not reliable as the reporter and selectable marker may not necessarily integrate in the same chromosomal location. Therefore the transfected cells could retain the selectable marker but lose the reporter. Accordingly, there is a need in the field of cancer therapeutics to develop methods for rapid screening of potential therapeutic compounds.

### SUMMARY OF THE INVENTION

The present invention provides compositions and methods for screening compounds which can affect the activity of thymidylate synthase. The screening method is based on the observation that TS can bind to TBEs in the mRNA for TS and interfere with its translation.

Accordingly, in one embodiment of the invention, a construct is provided which comprises from the 5′ to the 3′ direction of transcription, a promoter, a TBE cassette comprising TBE1 and TBE2 and a reporter gene. To obtain stably transfected cells, this construct also has a selectable marker.

In another embodiment, a method is provided for screening for compounds which reduce the activity of TS. The method comprises the steps of growing in the absence or presence of test compounds, cells comprising a DNA construct which comprises functionally joined together from the 5′ to 3′ direction of transcription, a promoter, a TBE cassette comprising TBE1 and TBE2, and a reporter gene; preparing extracts from the cell cultures and comparing the two extracts with respect to the level of reporter protein signal. An increase in the activity of the reporter protein signal is indicative of a compound which can reduce the TS activity.

In a yet another embodiment, a high throughput method for screening for compounds which reduce the activity of TS is provided. This method comprises the steps of growing in the absence or presence of test compounds, cells which have stably maintained a DNA construct which comprises functionally joined together from the 5′ to 3′ direction of transcription, a promoter, a TBE cassette comprising TBE1 and TBE2, and a reporter gene; preparing extracts from the cell cultures and comparing the two extracts with respect to the level of reporter protein signal. An increase in the activity of the reporter protein signal is indicative of a compound which can reduce the TS activity.

The screening methods can be carried out in multiwell format to provide greater screening capacity. Data is presented to demonstrate that known modulators of TS caused an increase in the reporter protein signal.

An advantage of this method is that compounds which reduce the activity of TS either directly (such as by reducing TS levels or activity) or indirectly (such as via modulation of a signal molecule which may affect the binding of TS to the TBEs) can be identified. Data is presented to demonstrate that a signal molecule known to be involved in the rTS signaling pathway, also caused an increase in the level of reporter protein signal.

In another embodiment, a stably transformed cell line is provided. This cell line is obtained by transfecting with a vector containing the DNA construct of the present invention. The cells can be used for screening of test compounds.

### BRIEF DESCRIPTION OF THE DRAWINGS

FIG. 1. Construction of pG3E1-2TBE-Neo. Key elements and restriction sites in the plasmids used for construction of pG3E1-2TBE-Neo are indicated.

FIGS. 2a-2c. Activity of the EGR-I promoter and TBEs in pGL3-basic plasmid. FIG. 2a—the EGR-I promoter was added upstream of the luciferase reporter gene of pGL3-basic plasmid as described in Example 1. The resultant construct pG3E1 was transfected into H630 cells and the luciferase assay was performed 24 hr later. Control: mock transfection; pG3E1: 5 μg of pG3E1 plasmid DNA per 2×10⁶ cells was used for transfection. RLU: relative light units. In FIGS. 2b and 2c, TS-binding elements were synthesized and added into pG3E1. The pG3E1-2TBE (5 μg) was transfected into six replicate aliquots of H630 cells (2×10⁶) and plated separately. After 24 hr, three of transfected samples were treated with 5-FU (10 μM) for an additional 24 hr (FIG. 2c), the cells were then processed for the luciferase assay according to the manufacturer's instructions. Control: mock transfection; pG3E1: transfected with pG3E1; pG3E1+T: transfected with pG3E1-2TBE only (FIG. 2b). P+T+FU: transfected with pG3E1-2TBE and 24 hr following transfection treated with 5-FU (FIG. 2c). Data are the average of triplicate experiments±S.D.

FIGS. 3a-3j. RLU production in H630-C6 cells in response to TS inhibitors. H630-C6 cells were evaluated for luciferase activity in response to treatment with the indicated compounds. The cells were exposed to the compounds at the indicated concentrations for 18 hr and luciferase assays were then performed according to the manufacturer's instructions. The mean values of triplicates or quadruplicates are shown along with the standard derivations. RLU: relative light units; CTL: control; 5-FU: 5-Fluorouracil (FIG. 3a); FUrd: 5-fluorouridine (FIG. 3b); FUdR: 5-fluoro-2′-deoxyuridine (FIG. 3c); TFT: trifluorothymidine (FIG. 3d); AG331: N6-[4-(morpholinosulfonyl)benzyl]-N6-methyl-2,6-diaminobenz-[c,d]-indole glucuronate (FIG. 3e); AG337: Nolatrexed (FIG. 3f); ZD1694: Raltitrexed, Tomudex (FIG. 3g); MTX: methotrexate (FIG. 3h); Act D: actinomycin D (FIG. 3i); AC: 5-azacytidine, G418: geneticin; TG: 8-thioguanosine (FIG. 3j).

FIGS. 4a and 4b. Evaluation of TS expression by Western blotting. H630-C6 cells were treated with the compounds as indicated for 18 hr. The cells were harvested for Western blots. TS: thymidylate synthase; 5-FU: 5-Fluorouracil; FUrd: 5-fluorouridine; FUdR: 5-fluoro-2′-deoxyuridine; TFT: trifluorothymidine; AG331: N6-[4-(morpholinosulfonyl)benzyl]-N6-methyl-2,6-diaminobenz-[c,d]-indole glucuronate; AG337: Nolatrexed; ZD1694: Raltitrexed, Tomudex; MTX: methotrexate; Act D: actinomycin D; AC: 5-azacytidine, G418: geneticin; TG: 8-thioguanosine.

FIGS. 5a and 5b. Evaluation of TS mRNA expression by RT-PCR. H630-C6 cells were exposed to the indicated compounds for 18 hr and total RNA was obtained for RT-PCR. Results are shown for 35 cycles of amplification. TS: thymidylate synthase; 5-FU: 5-Fluorouracil; FUrd: 5-fluorouridine; FUdR: 5-fluoro-2′-deoxyuridine; TFT: trifluorothymidine; AG331: N6-[4-(morpholinosulfonyl)benzyl]-N6-methyl-2,6-diaminobenz-[c,d]-indole glucuronate; AG337: Nolatrexed; ZD1694: Raltitrexed, Tomudex; MTX: methotrexate; Act D: actinomycin D; AC: 5-azacytidine, G418: geneticin; TG: 8-thioguanosine. Similar results were obtained at lower cycles (data not shown).

FIG. 6. A representation of the ability of acyl homoserine lactones to down-regulate TS in H630 cells.

FIG. 7. Induction of luciferase as a function of 3-oxo-6-hexanoyl-homoserine lactone concentrations. H630 cells containing pG3E1-2TBE-Neo were exposed to the indicated concentrations of 3-oxo-6-hexanoyl homoserine lactone for 18 hrs then analyzed for luciferase activity.

FIG. 8. Comparison of 5-fluorouracil (5-FU) and 3-oxo-hexanoyl-homoserine lactone on induction of luciferase. H630 cells containing pG3E1-2TBE-Neo were exposed to the indicated concentrations of each compound for 18 hrs and analyzed for luciferase activity.

### DETAILED DESCRIPTION OF THE INVENTION

This invention provides materials and methods for rapid screening of compounds that work specifically by reducing the activity of the enzyme thymidylate synthase (TS) either directly or indirectly. The term “TS activity” or “activity of TS” is used herein to mean both the enzyme activity of TS as well as the RNA binding activity of TS. Thus, this invention can be used for identifying compounds that directly or indirectly decrease or interfere with enzyme activity of TS or the RNA binding activity or TS or both. Such compounds have the potential for use as drugs to treat cancer and other diseases characterized by aberrant proliferation. The invention is based on the observation that the cancer chemotherapy target TS is also an mRNA binding protein that can bind to mRNAs through specific binding elements (TBEs) and inhibit protein synthesis by preventing translation of the mRNA. TS mRNA contains two different TBEs, one in the protein coding region and one in the 5′-untranslated region. The reason for two TBE's being present in TS mRNA is unknown.

The construct of the present invention comprises both distinct TBEs upstream of a reporter cDNA. The term “TBE cassette” as used herein means a sequence of DNA or RNA that comprises the sequence of TBE1 and TBE 2. A random sequence may be placed between the TBE elements. Sequences may also be placed at either ends of the TBE cassette to provide for suitable restriction sites. The term “TBE1” as used herein means any DNA or RNA that binds to TS in the manner similar to the naturally occurring TBE1 whereby translation of TS mRNA is interfered with and wherein the naturally occurring TBE1 is the region of mRNA in the 5′ noncoding region defined by the 36 nucleotide sequence in the human TS mRNA as described by Chu et. al., (Proc. Natl. Acad. Sci. USA 90:517-521, 1993). The term “TBE2” as used herein means any DNA or RNA that binds to TS in a manner similar to the naturally occurring TBE2 whereby translation of TS mRNA is interfered with and wherein the naturally occurring TBE2 is the region of mRNA defined by 70 nucleotide sequence in the human TS mRNA corresponding to nucleotides 480-550 (Lin et. al., Nucleic Acids Research 28: 1381-1389, 2000). It is well known in the art that minor changes in the particular sequence of the elements may be made without affecting their ability to bind to TS mRNA. In general, sequences having at least 90% homology can bind to the TS mRNA in a similar manner. Chu et al. provide several permissible changes in the sequence of TBE1 which do not affect its ability to bind to TS mRNA. The article of Chu et al. (incorporated herein by reference) and the knowledge of those skilled in the art will allow identification of permissive changes in both TBE1 and TBE2 sequences. For example, the sequence of TBE1 shown in SEQ ID NO:2 is 42 bases as opposed to the 36 nucleotide sequence in the naturally occurring TBE1 of Chu et al. (1993), and the sequence of TBE2 shown n SEQ ID NO:3 is 69 nucleotides as opposed to the 70 nucleotide sequence of naturally occurring TBE2 of Lin et al. (2000).

The presence of both TBE1 and TBE2 allows the use of this construct to screen for compounds that might affect the binding of TS to either TBE for a variety of reasons such as: modulation of gene products that interact with TS, modulation of gene products other than TS that bind to one or both TBEs, modulation of gene products that bind to TBEs and to TS, or modulation of gene products that affect the level of cellular TS. The plasmid of this invention may also include a selectable marker gene. The inclusion of such a gene makes the plasmid useful for study of agents in a wide variety of cell lines because it facilitates the development of stably transfected cells carrying the reporter.

The premise of this invention is that a plasmid containing a reporter gene (such as luciferase), two TBEs and a selectable marker that allows the isolation of cultured cells containing the plasmid can be used to identify compounds that disrupt TS binding to the reporter mRNA due to multiple mechanisms, some of which are not currently known. The invention will allow the identification of novel drugs that can affect TS levels and function by affecting pathways that regulate TS levels and activity. Examples of these mechanisms include TS stability, altered subcellular location, decreased synthesis and interactions with other proteins or RNA molecules. Since the assay incorporates the sensitive detection of light production by luciferase produced by cells containing the plasmid, the assay is suitable for the high throughput applications important for drug discovery in the pharmaceutical industry.

It should be noted that this invention allows for screening of compounds that reduce TS activity both directly by inhibiting TS as well as indirectly through interaction with other elements. An example of another gene that appears to affect expression and function of TS is the rTS gene[Chu, 1999; Chu, 2002; Black, 1996; Dolnick, 1997; Dolnick, 2000. The rTS gene produces an antisense RNA to TS pre-mRNA and mRNA as well as proteins that appear to affect TS expression and function. The expression of the rTS gene is thus another potential drug target within the cell, as altering rTS gene expression and function would affect TS function and expression. Thus in addition to the previously demonstrated use of a TBE-containing luciferase reporter to demonstrate the interaction of TS with TBEs within a cell, recombinant luciferase reporters containing TBEs can be used to identify novel compounds that alter TS levels or functions by previously unknown mechanisms, such as affecting rTS levels or activity.

The TBE cassette may contain more than one copy of either TBE or both. The TBEs may be placed in any relationship to each other. In a preferred embodiment, the TBE cassette contains only one copy of TBE1 and TBE2 separated by a random sequence of bases. While the sequences of TBE1 and TBE2 are provided herein, it should be noted that minor variations in the sequences which still allow it to bind to TS thereby enabling it to be used as a screening tool, are within the scope of this invention, as is the placement of the TBEs in reverse orientation.

The DNA construct of the present invention comprises a reporter gene operably linked to a promoter with a TBE cassette placed upstream of the reporter gene. Other well known elements such as a polyadenylation signal and one or more introns are also present. These are fairly conventional elements for expressing genes in plasmids. The promoter is placed to allow transcription and an intron/polyadenylation signal is placed to allow proper processing of the recombinant reporter RNA. In one embodiment, a selectable marker is also included. TBE1 (CCT CCG TCC CCC GCC CGC CGC GCC ATG CCT GTG GCC GGC TCG—SEQ ID NO:2) and TBE2 (G GAC TTG GGC CCA GTT TAT GGC TTC CAG TGG AGG CAT TTT GGG GCA GAA TAC AGA GAT ATG GAA TCA GAT T—SEQ ID NO:3) (and their copies if present) can be placed in any order. In one embodiment, a random sequence is placed separating them. In one embodiment, the random sequence is between 10 to 30 nucleotides long. In the exemplary plasmid in Example 1, the random sequence is 20 nucleotides. In addition, the TBEs can also be placed in a reverse orientation (GCT CGG CCG GTG TCC GTA CCG CGC CGC CCG CCC CCT GCC TCC—SEQ ID NO:4 for TBE1 and TTA GAC TAA GGT AGA GAG ACA TAA GAC GGG GTT TTA CGG AGG TGA CCT TCG GTA TTT GAC CCG GGT TCA GG—SEQ ID NO:5 for TBE2). The DNA construct may be part of a vector which may be a plasmid. One exemplary plasmid is described in Example 1.

The reporter gene is placed downstream of the TBE cassette. A “Reporter gene” means a gene encoding a protein that can be easily assayed, thereby providing a quantitative measure of the anount of protein (gene product) present. An example of a useful reporter gene that can be used in the DNA construct of the present invention is the firefly luciferase gene. The protein encoded by this gene catalyzes a reaction that produces light as one of its reaction products. The amount of emitted light can be easily quantitated and correlates with the amount of luciferase protein present. Another example of a useful reporter gene for this invention is the E. coli lacZ gene. The product of this gene, β-galactosidase, can be quantitated by a colorimeteric assay. A third example of a useful reporter gene according to the invention is the chloramphinicol acetyltransferase (CAT) gene, wherein the reaction products of the CAT enzyme can be conveniently assayed to provide a quantitative measure of the amount of enzyme present. A fourth example of a useful reporter gene is the green fluorescence protein gene, wherein the product can be quantitated by measuring the fluorescence. In general, these and other reporter genes are well known in the art and many of the constructs encoding these genes are commercially available.

The reporter is operably linked to a promoter. The term “promoter” refers to a DNA sequence located upstream of (i.e. 5′ to) the coding sequence of a gene which controls the expression of the coding sequence by providing a recognition and binding site for RNA polymerase. The promoter region may include additional recognition or binding sites for other factors involved in the regulation of transcription of the gene. Any promoter known in the art can be used. An example of a promoter used here is early growth response gene -1 (EGR-1) promoter. Other examples of suitable promoters include the CMV promoter (cytomegalovirus), the β-actin promoter, or an inducible promoter such as Tet-on which is inducible in suitably prepared cells by the addition of tetracycline or analogs such as doxycycline.

In the use of this method, cells can be transfected with the construct comprising the TBE cassette and the reporter gene operably linked to a promoter. In the case where the reporter gene is luciferase, in the presence of TS, the production of luciferase will decrease due to binding of TS to one or both TBEs. When these cells are exposed to candidate compounds which directly or indirectly reduce TS activity, luciferase production (hence the light produced) will increase. Accordingly, this invention provides a method for screening of potential compounds which can directly or indirectly reduce the activity of TS. To screen compounds, transfected cells are exposed to the compounds in varying concentrations and the signal produced (light) is measured at various times. An increase in the signal in indicative of a compound which reduces TS activity.

In one embodiment of the invention, a high throughput method is provided to screen for compounds that reduce the activity of TS either directly or indirectly. A convenient method of screening is to introduce into mammalian cells in culture a DNA construct comprising a TBE cassette, a reporter gene operably linked to a promoter and a selectable marker gene. The construct can be introduced by methods that are well known in the art including calcium phosphate method, electroporation, liposomes, retroviral infection and the like. The construct includes from the 5′ to the 3′ direction, a promoter, a TBE cassette, and a reporter gene. The reporter gene also contains other genetic elements well known in the art to be important for gene expression, such as a spliceable intron and a polyadenylation signal. The construct also includes the gene for a selectable marker. The selectable marker can be placed any where in relation to the two TBEs and the reporter gene. The expression of the selectable marker is required under certain culture conditions to maintain the introduced DNA in the cells. For example, if the selectable marker gene is the neomycin resistance gene and the cells are cultured in the presence of G418, only cells containing this marker gene would survive selection. Thus the introduced DNA can be stably maintained. Another example of a selectable marker is gene that provides resistance to hygromycin B. In this case, if cells are cultured in the presence of hygromycin B, only those cells which contain this marker gene would survive. In general, selectable markers are well known in the art and include genes that confer resistance to any cytotoxic or cytostatic agent.

Accordingly, in one embodiment a stably transformed cell line is provided. Stably transformed indicates that an exogenous nucleic acid has been introduced into the cell and is maintained in the cell or its progeny, if any, essentially for the life of the cell. Preferably the cell line is a mammalian cell line and more preferably the cell line is a human cell line. The selectable marker in the DNA construct of the present invention allows the establishment of stable cell lines that provide a convenient tool for screening compounds that modulate TS function and levels. To screen compounds, the stably transfected cells are grown in the presence of candidate compounds, lysed and the lysate (extract) is assayed for reporter gene activity. Control cultures can be grown in parallel in the absence of the test compound. The assay results for the reporter signal are then compared to determine if there is an increase in the reporter gene signal in the presence of the test compound. In the control cells not exposed to the test compounds, endogenous TS is expected to bind to TBEs and reduce the expression of the reporter signal. In the presence of a compound which reduces the ability of TS to bind to TBEs either directly or indirectly, the reporter signal production is increased. An increased reporter signal in the presence of a test compound, therefore, is indicative of its ability to reduce the activity of TS. The advantage of using stably transfected cells is that a DNA construct does not have to be introduced every time a potential compound is to be tested. Another advantage of the present construct is that the reporter and the selectable marker are in the same construct, thus avoiding problems associated with integration of different plasmids in different locations that can lead to loss of the desired gene even in cells that maintain the selectable marker. These features confer reproducibility and reliability of the method. For high throughput screening, a multiwell format can be utilized so that several test compounds can be evaluated at the same time.

The present invention also provides for kits for high throughput screening of compounds affecting TS activity. The kits include a DNA construct comprising a TBE cassette and a reporter gene operably linked to a promoter. Optionally, the kit may also include stably transfected cells and multiwell culture dishes for conducting the assays.

The invention is described further by way of examples presented below which are illustrative in nature and are not intended to be restrictive in any way.

### EXAMPLE 1

This example describes development of an exemplary plasmid. The abbreviations used are: 5-FU: 5-fluorouracil; AC: 5-azacytidine; Act D: actinomycin D; AG337: Nolatrexed; CH₂THF: 5,10-methylenetetrahydrofolate; dTMP: 2′-deoxythymidine-5′-monophosphate; dUMP: 2′-deoxyuridine-5′-monophosphate; EGR-I: early growth response gene; FBS: fetal bovine serum; FdUMP: 5-fluorodeoxyuridylate; FUdR: 5-fluoro-2′-deoxyuridine; FUrd: 5-fluorouridine; G418: geneticin; MTX: methotrexate; PBS: phosphate buffered saline; RLU: relative light units; TFT: trifluorothymidine; TG: 8-thioguanosine; ZD1694: Raltitrexed, Tomudex.

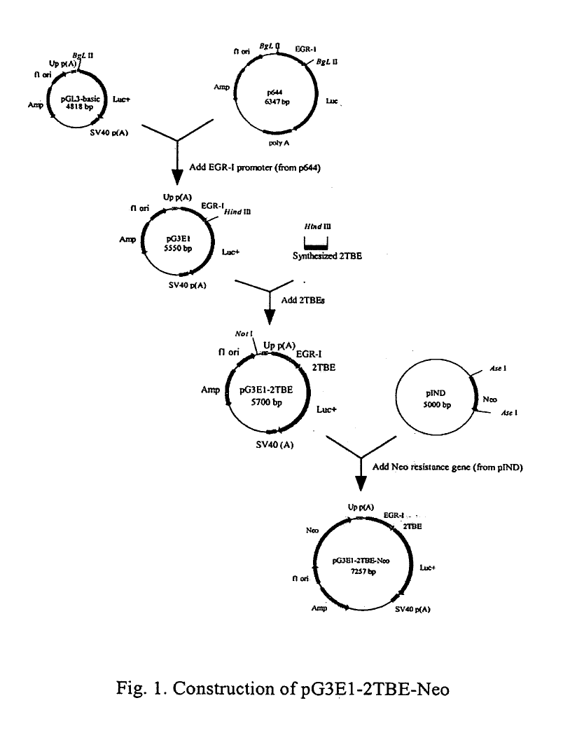

A plasmid (pG3E1-2TBE-Neo) was designed containing a luciferase reporter gene under control of an EGR-I promoter, both TBEs and a selectable marker suitable for use in mammalian cells (FIG. 1). The EGR-I promoter (from p644, a gift from Dr. Edward Chu, VA Cancer Center, Yale University, West Haven, Conn.) was added to Bgl II digested pGL3-basic (Promega, Madison, Wis.) to generate pG3E1. An oligonucleotide (150 bp) containing both TBE1 (TS mRNA UTR region and start site) and TBE2 (protein coding region) separated by a randomly chosen 20-nucleotide segment and flanked with Hind III sites was synthesized on an Applied Biosystems 381A DNA synthesizer. The sequence of this 2TBE unit is: 5′-GAT AAG CTT CCT CCG TCC CCC GCC CGC CGC GCC ATG CCT GTG GCC GGC TCG TCA GTC AGG CTA GCT ATA GCG GAC TTG GGC CCA GTT TAT GGC TTC CAG TGG AGG CAT TTT GGG GCA GAA TAC AGA GAT ATG GAA TCA GAT TAA GCT TGC-3′ (SEQ ID NO:1). The synthesized element was then amplified by PCR by using two 18-mer primers located at both terminals, and was cloned into the Hind III sites of pG3E1. This construct is designated as pG3E1-2TBE. The identity and orientation of the insert were confirmed by sequencing. To facilitate the selection of stable transformants, an Ase I fragment containing a neomycin-resistance gene (from pIND, Invitrogen) was blunt-ended and inserted down-stream of the luciferase reporter gene by ligation to pG3E1-2TBE that was linearized by digestion with Not I and blunt-ended using standard molecular biology techniques. The presence of all elements and orientation of cloned fragments was confirmed by sequencing and the final construct was termed pG3E1-2TBE-Neo (FIG. 1).

### EXAMPLE 2

This example describes the transient transfection of cells with the construct of the present invention. The human colon cancer cell line H630 (Park et al., 1987) was cultured in RPMI 1640 medium supplemented with 10% dialyzed fetal bovine serum (FBS) (Invitrogen, Grand Island, N.Y.). For transient transfection, pG3E1 or pG3E1-2TBE was electroporated into H630 cells (280 Volts, 600 μF and 13 Ohms, 5 μg plasmid DNA per 2×10⁶ cells) using a BTX ECM 600 electroporator (BTX Inc., San Diego, Calif.). After transfection, cells were cultured for 24 hr before assaying for luciferase activity.

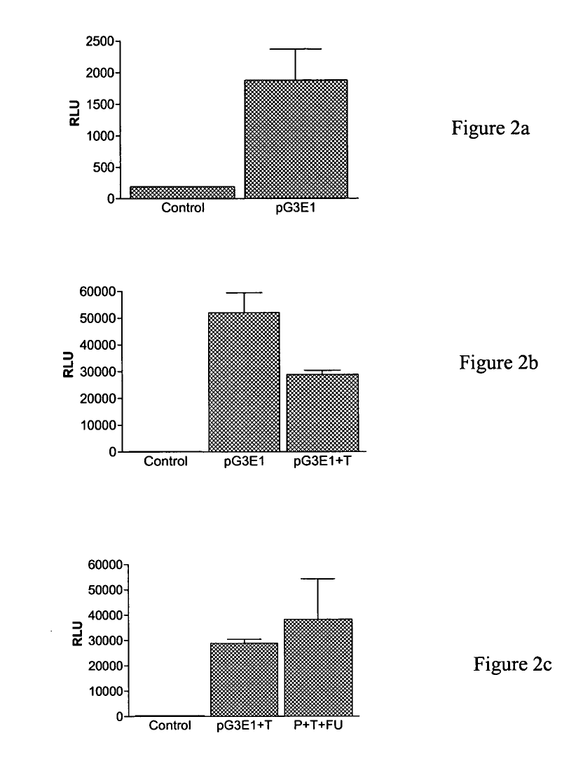

An approximate 10-fold increase of RLU was observed with pG3E1 compared to mock transfected cells (FIG. 2a). As shown in FIG. 2B the mock transfected control showed a RLU of 2.08±0.19×10²; the cells transfected with pG3E1 showed a RLU of 5.19±0.75×10⁴. The cells transfected with pG3E1-2TBE showed a RLU of 2.88±0.16×10⁴. Thus, consistent with the inhibition of luciferase production due to inclusion of the TBEs there was nearly a 2-fold decrease in the luciferase activity for the cells transfected with pG3E1-2TBE compared to pG3E1 (FIG. 2b). To evaluate a role for TS binding in the suppression of luciferase activity, transfected cells were exposed to the prodrug 5-FU (10 μM). This treatment resulted in a 33±13.8% increase in the RLU compared with untreated transfectants (FIG. 2c).

### EXAMPLE 3

This example describes the stable transfection of cells with the DNA construct of the present invention. To facilitate selection of stable transformants, we added a neomycin-resistance gene to the pG3E1-2TBE (FIG. 1) to generate a new plasmid termed pG3E1-2TBE-Neo as follows. For stable transfection, pG3E1-2TBE-Neo was linearized at the ampicillin resistance gene with Ase I and was introduced into H630 cells as described above. At 24 hrs post-transfection, the cells were exposed to 400 μg/ml of G418 for two weeks. Individual clones were picked at that time and screened for their ability to induce luciferase in response to treatment with 5-FU, a known TS inhibitor. Following selection with G418 and evaluation for changes in luciferase activity after exposure of the cells to 5-FU, the cloned subline H630/pG3E1-2TBE-Neo-L1-2-C6 (H630-C6) was chosen for further study.

The stably transfected H630-C6 cell line was maintained in RPMI 1640 plus 10% dialyzed FBS and 400 μg/ml of geneticin (G418). For luciferase assays, 2×10⁴ cells were seeded in each well of a 24-well plate in RPMI 1640 medium plus 10% FBS without G418 and were allowed to attach at least six hr, followed by drug exposure overnight (approximately 18 hr). The cells were then washed with PBS (137 mM NaCl, 2.7 mM KCl, 4.3 mM Na₂HPO₄, 1.4 mM KH₂PO₄, pH 7.4), lysed with Passive Lysis Buffer (Promega, Madison) and assayed for luciferase activity using the Luciferase Assay System (Promega, Madison, Wis.) according to the manufacturer's instructions. Relative light units (RLU) were measured with a Lumat LB 9501 luminometer (EG & G Berthold, Wildbad, Germany). All experiments were done in triplicate or quadruplicate with results presented as average±standard deviation.

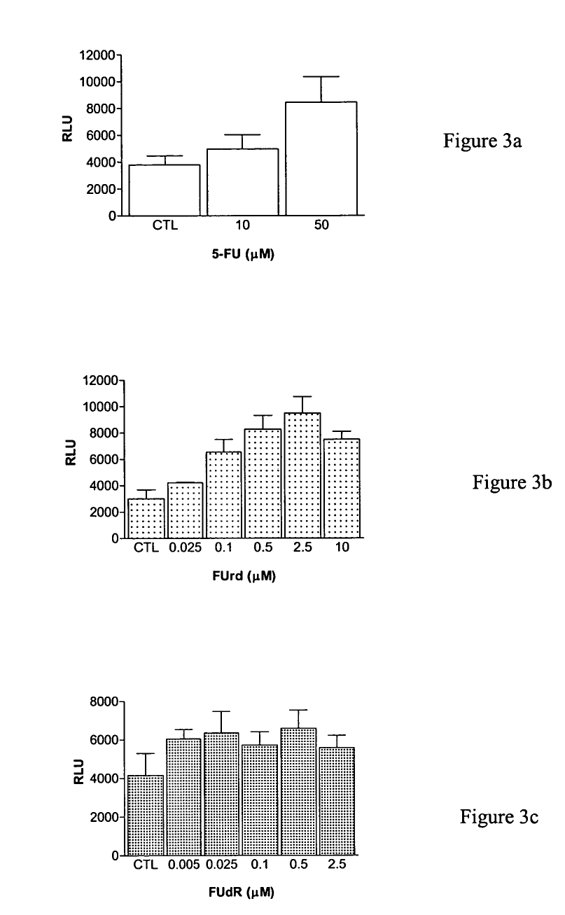

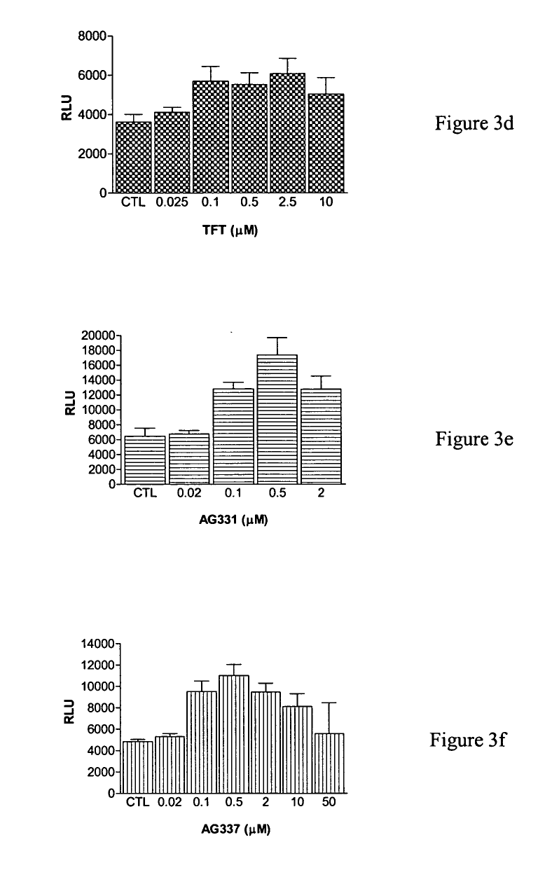

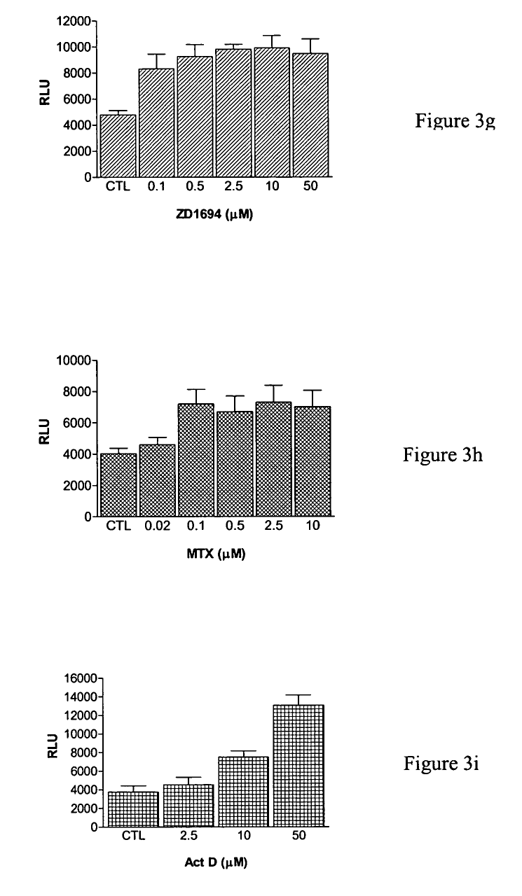

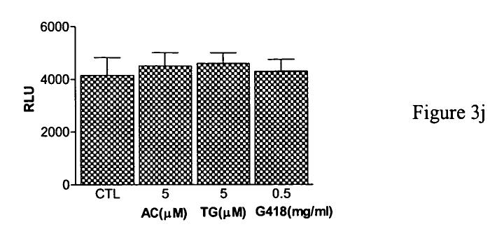

To test H630-C6 as a tool to identify TS modulators three different classes of compounds, i.e., specific or predominantly specific TS inhibitors, agents that inhibit folate pathways or inhibit TS as well as having other targets, and agents that act through TS unrelated sites of action were evaluated for their ability to induce changes in luciferase activity. Thus we tested major groups of compounds composed of pyrimidine TS prodrugs (5-FU, FUrd, FUdR, TFT), other direct and indirect TS inhibitors (AG331, AG337, ZD1694 and MTX) and cytotoxic compounds expected to work through pathways unrelated to TS (Act D, AC and TG). For inhibitors with primary sites of action against TS, we examined 5-FU, FUrd, FUdR, TFT, ZD1694, AG331 and AG337. Treatment of the cells with the three FdUMP prodrugs resulted in increases in RLU output that plateaued at concentrations that reflects the metabolic proximity of the prodrugs to FdUMP (FIGS. 3a-3d). These plateaus were 50 μM for 5-FU (higher concentrations not shown), 2.5 μM for FUrd and 5 nM for FUdR. TFT, a compound that is metabolized to a tight-binding but non-covalent inhibitor of TS reached a plateau at a concentration of 100 nM. The non-folate TS inhibitors AG331 and AG337 also caused concentration dependent increases in luciferase activity of up to 3- to 4-fold (FIGS. 3e and 3f). Interestingly, the TS-specific compound ZD1694 and the dihydrofolate reductase inhibitor MTX both caused increases in luciferase that were comparable despite having different major sites of action (FIGS. 3g and 3h). Both of these compounds require γ-polyglutamate addition within cells to become significant TS inhibitors although the tetrapolyglutamated ZD1694 (Kᵢ 1 nM) (Jackman and Calvert, 1995) is a much tighter inhibitor of TS than polyglutamated MTX (Kᵢ 13 μM) (Allegra et al., 1985). Surprisingly, when the cells were treated with Act D, a compound that does not target TS, a concentration-dependent increase of RLU was observed (FIG. 3i). However, as expected, no increase was observed when cells were exposed to AC, TG or G418, the compound used for selection of cells carrying the neo gene (FIG. 3j).

### EXAMPLE 4

This example describes the relationship between TS levels and luciferase production by various known TS inhibitors. Western blotting was performed as previously described (Dolnick and Black, 1996). Briefly, H630-C6 cells were washed with PBS and extracted with RIPA buffer. 10 μg of total protein was resolved by electrophoresis in a 10% SDS-PAGE and was blotted onto a PVDF membrane (Immobilon-P, Millipore, Bedford, Mass.). The membrane was hybridized with rabbit polyclonal antibody against TS (a gift from Dr. Edward Chu, VA Cancer Center, Yale University, West Haven, Conn.) diluted in SuperBlock Blocking Buffer (Pierce, Rockford, Ill.) plus 0.25% Tween 20 for one hour and hybridized with HRP-linked anti-rabbit secondary antibody (Jackson Laboratories) for an additional one hour. The membrane was detected with SuperSignal West Dura Extended Duration Substrate (Pierce, Rockford, Ill.) and was visualized by exposing the blot to Kodak XAR-5 film. For α-Tubulin, the membrane was hybridized with mouse monoclonal anti-α-tubulin, clone B-5-1-2 (Sigma, St Louis, Mo.) in 5% nonfat milk dissolved in Tris-buffered saline (10 mM Tris-HCl, pH 8.0, 150 mM NaCl) plus 0.1% Tween 20 (TBST), followed by HRP-linked anti-mouse secondary antibody IgG (Jackson Laboratories) in the same blocking buffer. The resulting signal was detected as described above.

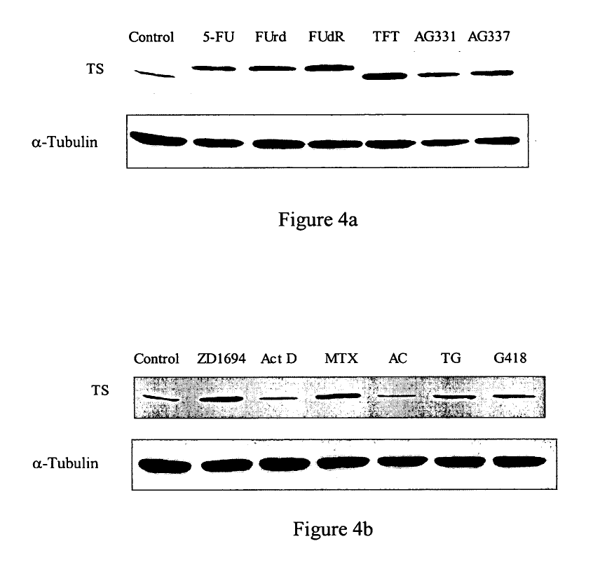

An increase in RLU production in response to treating the cells with TS inhibitors or their prodrugs is expected based upon previous demonstrations that several TS ligands can dissociate TS from the TBEs (Chu et al., 1994a, Lin et al., 2000). The induction of luciferase activity depends upon the ability to reduce the amount of endogenous TS bound to the TBEs in the recombinant luciferase transcripts. Dissociation of TS from the luciferase reporter mRNA is expected to be accompanied by the removal of TS from TBEs located in other mRNAs as well, such as endogenous TS mRNA. Therefore, an increase in TS is expected to accompany the increase in luciferase. To verify whether a translational mechanism is involved we evaluated the levels of TS from cells treated with the compounds described above by Western blotting. The concentration for each compound was chosen based upon the results obtained in the luciferase assays (FIG. 3). Elevated levels of TS were observed in the cells treated with compounds that were found to induce production of RLU in the luciferase assay with the exception of protein from cells treated with Act D (FIG. 4). 5-FU, FUrd and FUdR also altered the mobility of TS. This is due to the trapping of TS in a covalent ternary complex along with FdUMP, N^{5,10} methylene THF polyglutamate upon denaturation in preparation for SDS-PAGE, as previously observed (Drake et al., 1993).

As expected TG and AC, compounds that are metabolized to inhibitors of purine biosynthesis and nucleic acid precursors, did not alter TS protein levels, consistent with a lack of effect on luciferase activity, and the control compound, G418, was inactive as well (FIG. 3j). A notable exception to the observed increase in TS levels accompanying increased luciferase activity was found for Act D. Act D is known to inhibit RNA production but is not specifically associated with TS (Toku et al, 1983). Analysis of TS levels by Western blotting displays no increase of TS protein after Act D treatment (FIG. 4b)

### EXAMPLE 5

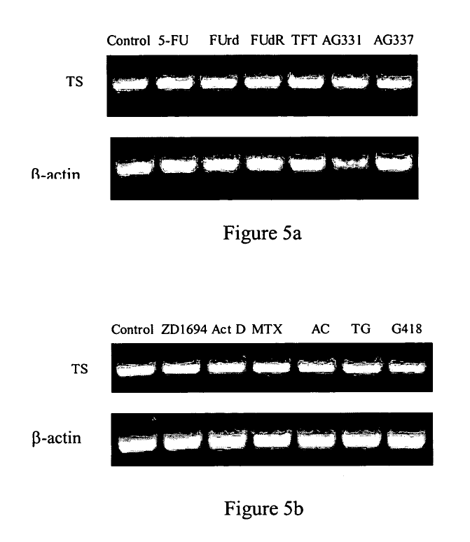

This example describes the TS mRNA levels in the presence of various TS inhibitors. RNA was prepared using an RNAqueous kit (Ambion, Austin, Tex.). Reverse transcription was done using a Cells-to-cDNA kit (Ambion, Austin, Tex.). Polymerase chain reaction (PCR) was performed as described previously (Dolnick et al., 1992). Primers used for TS mRNA are 5′-TTT GGA CAG CCT GGG ATT CTC-3′ (Sense—SEQ ID NO:6) and 5′-AAA GCA CCC TAA ACA GCC ATT-3′ (antisense—SEQ ID NO:7), generated a 600-bp PCR product. Primers used for β-actin are 5′-CAG CTC ACC ATG GAT GAT GAT A-3′ (sense—SEQ ID NO:8) and 5′-CCA GAC GCA GGA TGG CAT-3′ (antisense—SEQ ID NO:9), generated a 543-bp PCR product. The PCR cycling condition used was: 95° C. for 30 seconds, 60° C. for 30 seconds, 72° C. for 1 minute, repeated for 25, 30 and 35 cycles (for TS) or 20, 25 and 30 cycles (for β-actin). Product formation was evaluated by ethidium bromide staining and was resolved by electrophoresis in 1.5% agarose gels. Consistent with the Western blot results we did not detect any significant change of TS mRNA with any compound (FIG. 5).

The data presented in Examples 1-5 shows that the known potent TS inhibitors would induce luciferase activity, based upon their ability to bind to TS or serve as precursors to compounds that bind TS. All of these compounds induced luciferase activity relative to their TS binding ability. MTX, a compound with a primary site of action at dihydrofolate reductase, but one that can weakly inhibit TS after polyglutamate addition or can affect TS activity by depleting cells of reduced folate also stimulated luciferase activity. The ability of MTX to induce luciferase activity demonstrated the usefulness of the construct. However, it was surprising that MTX is almost as effective as ZD1694 in inducing luciferase activity, with both causing an approximately two-fold elevation at 100 μM (FIGS. 3g and 3h). This result obtained with MTX (and confirmed by a demonstration of elevated TS protein levels) indicates that the assay is useful for the identification of compounds that indirectly influence the activity or level of TS.

The ability of Act D to induce luciferase is surprising as this compound is known to be a potent inhibitor of mRNA synthesis, but does not have any obvious relationship to TS (Toku et al, 1983). Treatment of H630-C6 cells with Act D (50 μM), increased luciferase up to 3.5-fold (FIG. 3i), but did not induce TS levels indicating that its mechanism is not due to detachment of TS from its own mRNA. Act D is a known inhibitor of RNA polymerase and it seemed possible that there might be a differential effect on TS mRNA levels due to inhibition of synthesis that might lead to decreased TS mRNA and protein levels, which would also cause an increase in luciferase activity. A time course experiment examining the effects of Act D on TS levels as a function of time indicated TS levels dropped by 24 hr (data not shown), but not at 18 hr (FIG. 4) when the extracts were prepared for Western analysis. In confirmation of this interpretation, TS mRNA levels were also not altered in cells treated with Act D (FIG. 5). The mechanism by which Act D increases luciferase is not known.

### EXAMPLE 6

In addition to identifying compounds that influence TS indirectly, the assay can be implemented in a multiwell format that allows for the simultaneous testing of multiple compounds. The method for this is described using three separate RKO colon tumor cell lines containing the plasmid. The RKO cells were stably transfected as described for the H630 cells in Example 3.

A multiwell assay was performed in 24 and 48 well plate dishes. Those skilled in the art will recognize that this assay is applicable to dishes with more wells, allowing the screening of more compounds per assay by generating cells containing more copies of pG3E1-2TBE-Neo. For plates with either 24 or 48 wells, 5 to 10×10⁴ cells were seeded per well in RPMI 1640 medium supplemented with 10% fetal bovine serum was allowed to attach (6 hr). Test compounds were then added over a range of concentrations and the cells were incubated at 37° C. for approximately 18 hr in the presence of 5% CO₂. The cells were then washed with PBS and lysed with 100 μl Passive lysis buffer (Promega Corp., Madison, Wis.) for 15 min with agitation. Twenty μl of cell lysates were added in quadruplicate to a 96-well reading plate (Thermo Labsystems, Chantilly, Va.) and 100 μl of luciferase assay reagent (Promega) was injected into each well to start the assay. The assay window was set for a 2 sec delay after injection of the reagent and the samples were read for 10 sec. Values shown are the average of results obtained for quadruplicate wells. Results are shown in Table 1.

TABLE 1

| | RLU | | | RLU | | | RLU | | |
|---|---|---|---|---|---|---|---|---|---|
| [5-FU] | RKO | | | RKO | | | RKO | | |
| μM | clone 4 | % C | CV | clone 1 | % C | CV | clone 7 | % C | CV |
| 0 | 889 | 100 | 10.1 | 970 | 100 | 17.7 | 665 | 100 | 34.6 |
| 0.1 | 1078 | 121 | 15.1 | 1137 | 117 | 75.4 | 799 | 120 | 60.0 |
| 0.5 | 999 | 112 | 20.9 | 1314 | 135 | 41.2 | 1043 | 157 | 12.6 |
| 2.0 | 1214 | 137 | 18.2 | 1356 | 140 | 36.9 | 1119 | 168 | 7.68 |
| 10 | 1527 | 172 | 25.3 | 1738 | 179 | 43.9 | 1614 | 243 | 31.2 |
| 50 | 1559 | 175 | 11.5 | 1438 | 148 | 46.1 | 1560 | 156 | 13.6 |

Abbreviations: 5-FU, 5-fluorouracil; RLU, mean relative light units for quadruplicate samples; CV, coefficient of variation. % C is indicative of the effect relative to control i.e., without 5-FU and is calculated as [mean RLU sample × μM 5-FU/mean RLU 0 μM 5-FU] × 100. % C and CV were rounded to one decimal place.

As Table 1 illustrates, the assay can work with a cell line other than H630 and it can be used in a multiwell assay. In addition the results demonstrate that cloned cells containing the plasmid can be very sensitive to TS inhibitors. In the experiment shown in Table 1, changes in RLU can be detected with as little as 0.1 μM 5-fluorouracil, a very low concentration for this drug. The increase in RLU from approximately 20 to 75% over the wide concentration of drug used (0.1 to 50 μM) also demonstrates that the assay is well suited to identify compounds that might have different activities in altering the amounts or activity of TS within cells.

### EXAMPLE 7

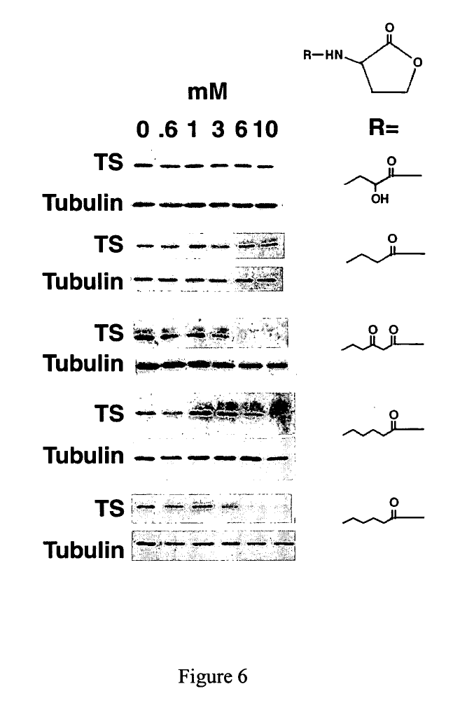

The signaling molecules produced by the rTS signaling pathway cause a decrease in the amount of TS found in cells. These signaling molecules can simulate the effects of the compound 3-oxo-hexanoyl homoserine lactone in activating the receptor for this compound when it is present in a recombinant bacteria. This result suggested the compound itself might be able to down-regulate the amounts of TS in cells. This hypothesis was validated by the results of an experiment portrayed in FIG. 6. Accordingly, the down-regulation of TS in H630 (human colon cancer) cells treated with acyl-homoserine lactones was tested. Cells growing in RPMI 1640 culture medium, supplemented with 10% dialyzed fetal bovine serum at 37° C. in an atmosphere of 95:5 air:CO₂. The compounds with the structures shown were added to the cells and allowed to incubate overnight (approximately 16 hours). The cells were extracted and TS measured by Western blotting as described. The compounds from top to bottom are: (1) 3-hydroxy butanoyl homoserine lactone, (2) butanoyl homoserine lactone; (3) 3-oxo-hexanoyl homoserine lactone; (4) d, 1-hexanoyl homoserine lactone, and (5) 1-homoserine lactone. Tubulin is shown as a loading standard. As FIG. 6 shows, the 3-oxo-hexanoyl homoserine lactone is the most active of all the compounds shown. The effect of the compounds that have activity is specific since the 1-hexanoyl compound is twice as active as the racemic mixture (d, 1), showing that only the 1-enantiomer is active and the d-enantiomer is inactive. The high concentrations required to observe an effect are of no significance, since acyl homoserine lactones with acyl side chains of the length shown are very unstable under the conditions used, and therefore are useful for defining structural requirements for activity for future compounds that might be more stable (Yates et al., 2002).

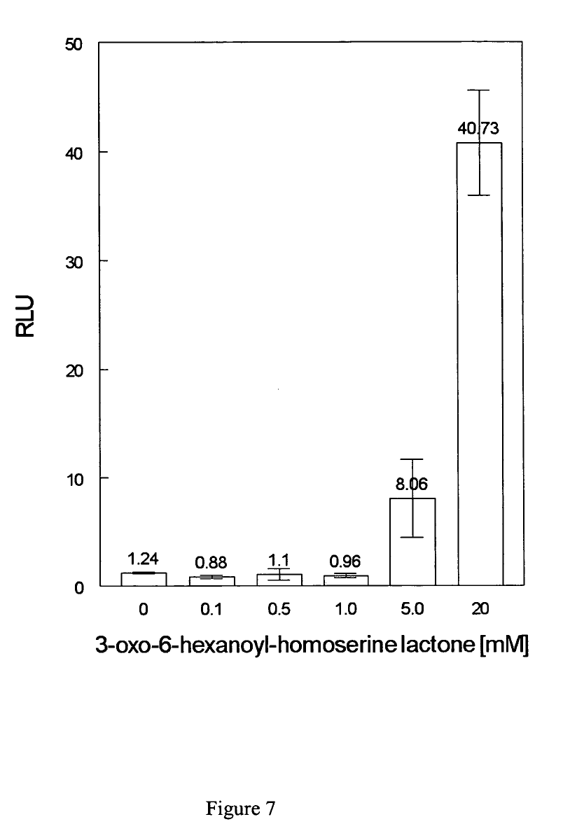

Based on the results shown in FIG. 6, experiments were carried out to determine if the method of the present invention could identify the 3-oxo-hexanoyl homoserine lactone, which was demonstrated as down regulating the amount of TS, as an efficient inducer of light at the concentrations at which TS is down-regulated. H630 cells containing pG3E1-2TBE-Neo were exposed to the indicated concentrations of 3-oxo-6-hexanoyl homoserine lactone for 18 h then analyzed for luciferase activity (FIG. 7). The results indicate that increasing the concentration of 3-oxo-hexanoyl homoserine lactone increased the luciferase activity. These results demonstrate that the present invention can be used to screen for compounds that directly or indirectly affect TS activity.

### EXAMPLE 8

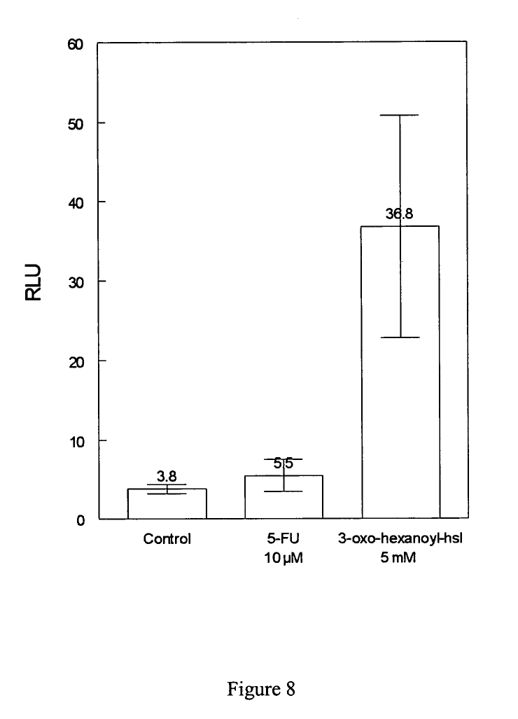

In another illustration of this invention, the test compound, 3-oxo-6-hexanoyl- was compared to 5-fluorouracil, a known inhibitor of TS that does not down-regulate the amount of TS in cells but induces expression of luciferase in the assay. As shown in FIG. 8, a significant effect of the 3-oxo-6-hexanoyl-homoserine lactone was observed as compared to 5-FU. The result demonstrates that the acyl-homoserine lactone, thought to be a mimic of the signals produced by the rTS signaling pathway, induces recombinant luciferase activity from an mRNA with two TBEs approximately 6 to 7-fold more than the TS inhibitor 5-FU. The result demonstrates the better sensitivity of the assay to TS modulators than a well known TS inhibitor and anticancer drug.

It will be recognized by those skilled in the art that the examples presented here and in the Appendix are illustrative and routine modifications to the method and materials described above are intended to be within the scope of the invention as described herein.

### References

Black, A. R. and B. J. Dolnick (1996). “Expression of rTS correlates with altered growth regulation of thymidylate synthase.” Cancer Res 56(4): 700-705.

Chu, E. and C. J. Allegra (1996). “The role of thymidylate synthase as an RNA binding protein.” Bioessays 18(3): 191-8.

Chu, E., T. Cogliati, et al. (1996). “Identification of in vivo target RNA sequences bound by thymidylate synthase.” Nucleic Acids Res 24(16): 3222-8.

Chu, E., S. M. Copur, et al. (1999). “Thymidylate synthase protein and p53 mRNA form an in vivo ribonucleoprotein complex.” Mol Cell Biol 19(2): 1582-94.

Chu, E., T. Takechi, et al. (1995). “Thymidylate synthase binds to c-myc RNA in human colon cancer cells and in vitro.” Molecular and Cellular Biology 15: 179-185.

Chu, E., D. Voeller, et al. (1993). “Identification of an RNA binding site for human thymidylate synthase.” Proc Natl Acad Sci USA 90(2): 517-21.

Chu, E., D. M. Voeller, et al. (1991). “Autoregulation of human thyrnidylate synthase messenger RNA translation by thymidylate synthase.” Proceedings of the National Academy of Sciences, USA 88: 8977-8981.

Chu, E., D. M. Voeller, et al. (1994). “Identification of a thymidylate synthase ribonucleoprotein complex in human colon cancer cells.” Mol Cell Biol 14(1): 207-13.

Chu, J. (1999). Natural antisense (rTSalpha) RNA mediated down-regulation of thymidylate synthase gene expression. Molecular Pharmacology and Cancer Therapeutics. Buffalo, N.Y., SUNY at Buffalo.

Chu, J. and B. J. Dolnick (2002). “Regulation of thymidylate synthase RNA levels by naturally occurring antisense RNA.” Biochim et Biophysica Acta: In Press.

Danenberg, P. V. (1977). “Thymidylate Synthase: A target enzyme in cancer chemotherapy.” Biochemistry Biophysica Acta 473: 73-79.

Dolnick, B. J., Zhang, Z.-G., Hines, J. D., and Rustum, Y. M. (1992) “Quantitation of dihydrofolate reductase and thymidylate synthase mRNAs in vivo and in vitro by polymerase chain reaction.” Oncology Research 4: 65-72.]

Dolnick, B. J. (2000). “Regulation of thymidylate synthase mRNA levels by RNA editing.” Proceedings AACR 41: 945.

Dolnick, B. J., K. Lu, et al. (1997). “Recent advances in the study of rTS proteins. rTS expression during growth and in response to thymidylate synthase inhibitors in human tumor cells.” Adv Enzyme Regul 37: 95-109.

Johnson, L. F. (1994). “Posttranscriptional regulation of thymidylate synthase gene expression.” Journal of Cellular Biochemistry 54: 387-392.

Leichman, C. G., H.-J. Lenz, et al. (1997). “Quantitation of intratumoral thymidylate synthase expression predicts for disseminated colorectal cancer response and resistance to protracted-infusion fluorouracil and weekly leucovorin.” Journal of Clinical Oncology 15: 3223-3229.

Lenz, H.-J., C. G. Leichman, et al. (1995). “Thymidylate synthase mRNA level in adenocarcinoma of the stomach: A predictor for primary tumor response and overall survival.” Journal of Clinical Oncology 14: 176-182.

Li, W., J. Fan, et al. (1995). “Lack of functional retinoblastoma protein mediates increased resistance to antimetabolites in human sarcoma cell lines.” Proc Natl Acad Sci USA 92(22): 10436-40.

Lin, X., L. A. Parsels, et al. (2000). “Characterization of a cis-acting regulatory element in the protein coding region of thymidylate synthase mRNA.” Nucleic Acids Res 28(6): 1381-9.

Toku, S., Nabeshima, Y., and Ogata, K. (1983) “Effects of low dose actinomycin D treatment in vivo on the biosynthesis of ribosomal protein in rat liver.” Journal of Biochemistry (Tokyo) 93:349-359.]

Yates, E. A., Philipp, B., Buckley, C., Atkinson, S., Chhabra, S. R., Sockett, R. E., Goldner, M., Dessaux, Y., Camara, M., Smith, H., and Williams, P. (2002) “N-acylhomoserine lactones undergo lactonolysis in a pH-, temperature-, and acyl chain length-dependent manner during growth of Yersinia pseudotuberculosis and Pseudomonas aeruginosa.” Infection and Immunity 70: 5635-5646.

1. A method for identifying a compound which reduces the activity of thymidylate synthase comprising the steps of:
growing in the absence of a test compound a first culture of mammalian cells comprising a DNA construct comprising functionally joined together in the 5′ to 3′ direction of transcription, (i) a promoter, (ii) a TBE cassette comprising at least one copy of TBE 1 in the forward orientation (SEQ ID NO: 2) or reverse orientation (SEQ ID NO: 4) and at least one copy of TBE2 in the forward orientation (SEQ ID NO:3) or reverse orientation (SEQ ID NO:5); and (iii) a reporter gene; wherein the TBE cassette and the reporter gene are operably linked to the promoter.
growing a second culture of the cells in the presence of the test compound;
lysing the first culture and the second culture to produce a first lysate and a second lysate respectively;
assaying the first lysate and the second lysate for activity of the protein encoded by the reporter gene; and
comparing the activities of the first lysate and the second lysate, wherein an increase in the activity in the second lysate is indicative of a compound which reduces the activity of thymidylate synthase.

2. The method of claim 1, wherein the at least one copy of TBE1 and the at least one copy of TBE2 are separated by a DNA spacer of about 20 nucleotides.

3. The method of claim 1, wherein the reporter gene is luciferase gene.

4. The method of claim 1, wherein the promoter is the ERG-1 promoter.

5. The method of claim 1, wherein the TBE cassette has the sequence of SEQ ID NO:1.

6. The method of claim 1, wherein the DNA construct is contained in a vector.

7. The method of claim 6, wherein the vector containing the DNA construct is plasmid pG3E1-2TBE.

8. A method for identifying a compound which reduces the activity of thymidylate synthase comprising the steps of:
growing in the absence of a test compound a first culture of mammalian cells comprising a DNA construct comprising functionally joined together in the 5′ to 3′ direction of transcription (i) a promoter; (ii) a TBE cassette comprising at least one copy of TBE1 in the forward orientation (SEQ ID NO:2) or reverse orientation (SEQ ID NO:4) and at least one copy of TBE2 in the forward orientation (SEQ ID NO:3) or reverse orientation (SEQ ID NO:5); and (iii) a reporter gene, the TBE cassette and the reporter gene being operably linked to the promoter, wherein the DNA construct is stably maintained in the cells;
growing a second culture of the cells in the presence of the test compound;
lysing the first culture and the second culture to produce a first lysate and a second lysate respectively;
assaying the first lysate and second lysate for activity of the protein encoded by the reporter gene; and
comparing the activities of the first lysate and the second lysate, wherein an increase in the activity in the second lysate is indicative of a compound which reduces the activity of thymidylate synthase.

9. The method of claim 8, wherein the at least copy of TBE1 and the at least one copy of TBE2 are separated by a DNA spacer of about 20 nucleotides.

10. The method of claim 8, wherein the reporter gene is luciferase gene.

11. The method of claim 8, wherein the promoter is the ERG-1 promoter.

12. The method of claim 8, wherein the TBE cassette has the sequence of SEQ ID NO:1.

13. The method of claim 8, wherein the DNA construct is contained in a vector.

14. The method of claim 13, wherein the vector containing the DNA construct is plasmid pGE31-2TBE1-Neo.

15. A DNA construct comprising:
functionally joined together in the 5′ to 3′ direction of transcription, (i) a promoter, (ii) a TBE cassette comprising at least one copy of TBE 1 in the forward orientation (SEQ ID NO: 2) or reverse orientation (SEQ ID NO: 4) and at least one copy of TBE2 in the forward orientation (SEQ ID NO:3) or reverse orientation (SEQ ID NO:5); and (iii) a reporter gene; wherein the TBE cassette and the reporter gene are operably linked to the promoter.

16. The method of claim 15, wherein the at least copy of TBE1 and the at least one copy of TBE2 are separated by a DNA spacer of about 20 nucleotides.

17. The construct of claim 15, wherein the reporter gene is luciferase gene.

18. The construct of claim 15, wherein the promoter is the ERG-1 promoter.

19. The construct of claim 15, wherein the TBE cassette has the sequence of SEQ ID NO:1.

20. The construct of claim 15, wherein the DNA construct is contained in a vector.

21. The construct of claim 20, wherein the vector containing the DNA construct is plasmid pGE1-2TBE.

22. The construct of claim 15 further comprising a selectable marker.

23. The construct of claim 22, wherein the selectable marker is neomycin resistance gene.

24. The construct of claim 23, wherein the DNA construct is contained in the plasmid pG3E1-2TBE-Neo.

25. Mammalian cells stably transfected with a vector comprising a DNA construct comprising:
functionally joined together in the 5′ to 3′ direction of transcription, (i) a promoter, (ii) a TBE cassette comprising at least one copy of TBE 1 in the forward orientation (SEQ ID NO: 2) or reverse orientation (SEQ ID NO: 4) and at least one copy of TBE2 in the forward orientation (SEQ ID NO:3) or reverse orientation (SEQ ID NO:5); and (iii) a reporter gene; wherein the TBE cassette and the reporter gene are operably linked to the promoter; and
a selectable marker gene.

26. The cells of claim 25, wherein the cells are human cells.

27. The cell of claim 26, wherein the cells are H630 colon tumor cells or RKO colon tumor cells.

28. The cells of claim 24, wherein the vector is the plasmid pG3E1-2TBE-Neo.
